(19) United States
(12) Patent Application Publication
(10) Pub. No.: US 2021/0259761 A1
(43) Pub. Date: Aug. 26, 2021

(54) SELECTIVE RESECTION AND DETECTION OF TISSUE MASS

(71) Applicant: HERACURE MEDICAL LTD., Even Yehuda (IL)

(72) Inventor: Gal Meister, Even Yehuda (IL)

(73) Assignee: HERACURE MEDICAL LTD., Even Yehuda (IL)

(21) Appl. No.: 16/973,872

(22) PCT Filed: Jun. 12, 2019
(86) PCT No.: PCT/IB2019/054902

§ 371 Date: Dec. 10, 2020

Related U.S. Application Data
(60) Provisional application No. 62/684,214, filed on Jun. 13, 2018.

(51) Int. Cl. A61B 18/14; A61B 17/32

(52) U.S. Cl. CPC A61B 18/14; A61B 17/320068; A61B 2217/005; A61B 2018/00577; A61B 2217/007; A61B 2017/32007; A61B 2017/320069; A61B 2018/1435

(57) ABSTRACT

A system includes a cutting portion, an actuator coupled to the cutting portion for moving the cutting portion, a controller coupled to the actuator, and a sensor in communication with the controller. The sensor senses if tissue contacted by the cutting portion has hardness above a threshold. If the hardness is above the threshold, the controller permits cutting of the tissue and if the hardness is not above the threshold, the controller does not permit cutting of the tissue. Conversely, the system can have a mode of operation in which if the hardness is below the threshold, the controller permits cutting of the tissue and if the hardness is not below the threshold, the controller does not permit cutting of the tissue.

### FIELD OF THE INVENTION

The present invention related generally to the field of medical devices, and more particularly, to a system for resection of tissue, such as a system that distinguishes between hard and soft tissue.

### BACKGROUND OF THE INVENTION

There are various instances in which it may become necessary to resect a segment of a patient's tissue or muscle, for example, to remove harmful or potentially harmful benign growths or cancerous tissue segments, or to obtain tissue samples, while avoiding complications such as unintended perforation or damage to the surrounding tissue or organs. For example, uterine wall perforation is a known complication of intrauterine device insertion and navigation via a vaginal approach, resulting in the device being totally or partially in the abdominal cavity, which requires immediate surgical repair.

There are also various instances in which it may be necessary to perform tissue ablation for large tumors, fibroids or lesions, while the penetration site to the tissue is constrained in dimension. RF energy may be delivered to diseased regions (e.g., tumors) for the purpose of ablating predictable volumes of tissue with minimal patient trauma; however, if a large area of tissue is needed to be ablated with minimal consuming time, there is a need to generate a larger volumetric shape of electrodes to reduce treatment time and complexity. One drawback of tissue ablation using RF electrodes is when the electrodes are not properly located and fixed in the tissue, thus generating heat damage to the surrounding healthy tissue or organs. Therefore, there is a need for a simple system that can be navigated to penetrate and hold the electrodes in the target tissue in high precision, while enabling high volumes of tissue ablation, sometime even 8-10 fold of ablated volume compared to the initial device penetration diameter.

A good candidate disease for such treatment is the uterine fibroid. Uterine fibroids (leiomyomata uteri) are benign solid hard tumors that affect the majority of women in the USA by age 50. While often asymptomatic, fibroids can result in abnormal uterine bleeding, pelvic pressure, subfertility, dyspareunia, and other symptoms. Uterine fibroids are the leading indication for hysterectomy in the USA, Europe, and other countries. While treatment options (hysterectomy, myomectomy, uterine artery embolization) exist, they typically involve major surgery and inpatient admission, require incisions and general anesthesia, and can be associated with significant adverse events and prolong the return to the activities of daily living. Therefore there is a need for a safe and effective system for the resection of deep and large fibroids (3-10 cm in diameters) in minimally invasive for women who desire uterine conservation.

### SUMMARY OF THE INVENTION

The present invention seeks to provide systems and method for selective resection of tissue of different stiffnesses. The system distinguishes between soft tissue and hard tissue mass based on physical features and selectively resects and removes the undesired tissue mass, fibroid, lesion, or tumor from a patient's body.

In one embodiment, the medical device includes a cutting portion with blades that vibrates at a certain frequency (without limitation, in the range of 100-100,000 oscillations per minute, or in the ultrasonic range) for small and limited stroke distance or angle (without limitation, in the range of 0.05 mm-5 mm). When the cutting portion of the system comes in contact with soft/flexible/low stiffness tissue, the tissue is flexible enough to deform without being cut. This is due to the limited/small amplitude of the cutting stroke/distance/angle exerted on the tissue. Due to the flexibility of the tissue, no solid resistance is generated on the cutting portion, making resection of the soft tissue impossible. However, when the cutting portion of the system comes into contact with the rigid/hard fibroid or lesion, the hard tissue cannot deform because the fibroid resists the movement of the cutting portion, thereby allowing the cutting portion to easily resect/penetrate the hard tissue, fibroid, lesion, or tumor. Thus the invention provides a safer system for cutting hard tissue, fibroids, lesions or tumors while avoiding damage to the surrounding tissues.

The inability of the cutting portion to cut soft tissue is a factor of various parameters, including, without limitation, the vibration frequency and amplitude, and the shape and the size of the cutting portion/blades, and the vacuum level applied on the tissue.

There is provided in accordance with an embodiment of the invention, a system includes a cutting portion with a certain geometry, an actuator coupled to the cutting portion for moving the cutting portion, and a controller coupled to the actuator. The actuator oscillates the cutting portion at a certain frequency that enables to cut tissue with hardness above threshold.

In yet another embodiment, a system may include a cutting portion, an actuator coupled to the cutting portion for moving the cutting portion, a controller coupled to the actuator, and a sensor in communication with the controller. The sensor senses if tissue contacted by the cutting portion has hardness above a threshold. If the hardness is above the threshold, the controller permits cutting of the tissue and if the hardness is not above the threshold, the controller does not permit cutting of the tissue. Conversely, the sensor may sense if tissue contacted by the cutting portion has hardness above threshold by detecting a “no resection” condition in cases the cutting portion is not able to resect the soft tissue. Detection of “no resection” condition may be based on physical parameters of the system such as, but not limited to, an increase of load/force on the resection portion, an increase load/force on the actuator, an increase of load on the suction system, an increase/decrease of ultrasonic resonance frequencies, and more. Conversely, the system can have a mode of operation in which if the hardness is below the threshold, the controller permits cutting of the tissue and if the hardness is not below the threshold, the controller does not permit cutting of the tissue.

In yet another embodiment, the system may include an additional interference system/unit that can reject/push tissue not intended for cutting, away from the resection portion, and resume the resection process from the beginning.

In addition, the present invention seeks to provide systems and method for preventing unintended tissue perforation during the initial deployment of the device. Having unintended tissue perforation during a routine procedure is a complication that requires additional surgery repair and thus must to be avoided. For example, uterine wall perforation is a known complication of intrauterine device insertion via vaginal approach, resulting with device totally or partially in abdominal cavity, which require immediate surgery repair.

There is provided in accordance with an embodiment of the invention a surgical device including a housing formed with a window, a cutting element disposed in the housing and coupled to a vibration source, the vibration source operative to cause the cutting element to oscillate, an imaging sensor, and an actuator coupled to the housing or to the cutting element and in operative communication with the imaging sensor.

The actuator may align the window with an imaging direction of the imaging sensor. A directional suction source may draw tissue into the housing, and the actuator may align the directional suction source with the plane of an imaging direction of the imaging sensor. The cutting element may be movable in a linear motion or a non-linear motion. The housing may be coupled to a rotatable helical element, wherein rotation of the helical element causes linear movement of the housing.

The helical element may be coupled to a bendable member, such that the helical element is movable along a non-linear path in response to bending of the bendable member.

A tissue hardness detector may be coupled to the vibration source. For example, if hardness is not above a threshold, the vibration source decreases a vibration amplitude so as not to permit tissue cutting. For example, if hardness is not above a threshold, the tissue hardness detector actuates an interference device that interferes with the suction source and does not permit the suction source to draw tissue into the housing. The interference device may include a solenoid that injects liquid or pressurized gas that opposes suction of the suction source so as to eject the tissue away from the housing.

The helical element may be slidable over a shaft coupled to the cutting portion. Helices of the helical element may be expandable radially outwards.

The helical element may be coupled to the imaging sensor or to another imaging sensor.

An actuator sensor may be provided. The sensor may measure a change or deflections in a vacuum load level of the cutting element, or it may measure a change in a load of the actuator, or it may measure a change in mass flow at or near the cutting element, or it may measure a difference between forces, deflections or power of the cutting element compared to forces, deflections or power of the actuator.

There is provided in accordance with an embodiment of the invention surgical device including a helical cutting element disposed around an oscillatory cutting element.

The helical cutting element may rotate about a rotation axis which is either collinear with or parallel to a longitudinal axis along which the oscillatory cutting element oscillates.

The helical cutting element may be movable linearly with respect to the oscillatory cutting element from a position proximal to the oscillatory cutting element to a position that overlies the oscillatory cutting element, and to a position distal to the oscillatory cutting element.

There is provided in accordance with an embodiment of the invention a system for ablation including a helical member coupled to a housing member and configured to move and position the housing member in a tissue, a portion of the helical member having a side aperture, and a flexible member deployable through the side aperture, the flexible member being capable of assuming a helical shape and transmitting RF energy to the tissue.

The flexible member may have a variable cross section. A portion of the helical member may be coupled to a bendable member, wherein the bendable member is operative to cut a route in the tissue in accordance with bending of the bendable member. An actuator may be coupled to the helical member and be in operative communication with an imaging sensor, wherein the actuator is operative to align the helical member with an imaging direction of the imaging sensor.

Also the present invention seeks to provide systems and method for RF ablating of large tissue area with adjustable shape to meet the actual shape of the tumor, lesion, fibroid. Various types of RF electrodes were designed to be expandable; however, no system exists for combining tissue penetration and holding technique with small crossing profile together with up to 10-fold expandable RF ablation volume.

### BRIEF DESCRIPTION OF THE DRAWINGS

The present invention will be better understood with reference to the following drawings and description. The components in the figures are not necessarily to scale, emphasis instead being placed upon illustrating the principles of the invention.

FIG. 1 is a simplified flow chart illustration of the system methodology to selectively resect hard tissue mass, while avoiding the resection of soft tissue mass.

FIG. 2 is a simplified flow chart illustration of the system methodology to detect, recognize and selectively resect tissue mass of type A, while avoiding the resection of tissue type B, including a mechanism of interference to eject tissue that are not intended for resection.

FIG. 3 are simplified pictorial illustrations of one embodiment of such a system, including a controller connected to one or more of the following components (but not limited to): resection device, vacuum/aspiration source, interference system, fluid management, RF generator, and foot pedal.

FIGS. 4A-4C are simplified pictorial illustrations of one example of a resection device.

FIG. 5 is a simplified pictorial illustration of system mechanism to rotate and vibrate the resection blade while performing tissue aspiration.

FIGS. 5A and 5B are simplified illustrations of the resection device tube rotating without a swivel suction port rotating.

FIGS. 6-6E are simplified pictorial illustrations of a corkscrew (helical) element and bendable leaf to prevent unintended tissue perforation during the initial deployment of the device.

FIG. 7 is a simplified pictorial illustration of additional shapes and designs of the leaf element 107.

FIG. 8 is a simplified pictorial illustration of yet another design, allowing bending and flexibility of the resection device to treat surrounding tissues.

FIG. 9 is simplified pictorial illustrations of the mechanism to retract and rotate the corkscrew element 106 (of FIG. 6) in a safe manner.

FIGS. 10-12 are simplified pictorial illustration of design of an orientation fixture 130 to align the boresight of the device shaft 100 with the imaging system 200.

FIGS. 13-15B are simplified pictorial illustrations of a steerable corkscrew element 106 attached to flexible shaft 108.

FIGS. 16-17 are simplified pictorial illustrations of an RF ablation device with an expandable RF electrode.

FIGS. 18A-18D are simplified pictorial illustrations of the advancement sequence of the expandable RF electrode.

FIGS. 19A-19D are simplified pictorial illustrations of yet another design of the expandable electrodes.

FIGS. 20A-20B are simplified pictorial illustrations of combinations of plural expandable electrodes of various shapes.

### DETAILED DESCRIPTION OF EMBODIMENTS

Reference will be now be made in detail to embodiments of the present disclosure, an example of which is illustrated in the accompanying drawings. The term “distal” refers to a direction that is generally towards a target site within a patient's anatomy during a medical procedure. The term “proximal” refers to a direction that is generally towards a physician during a medical procedure.

In one aspect, the system of the invention can perform minimally invasive procedures in a body of a patient, such as for transcervical removal of intramural and subserosal uterus fibroids. A handle may be provided or the device may be connected to some other manipulating tool.

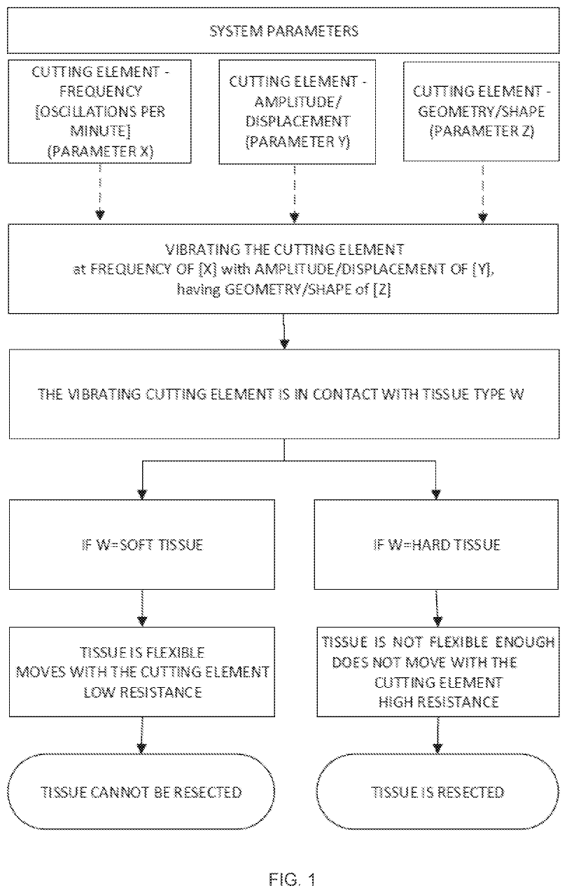

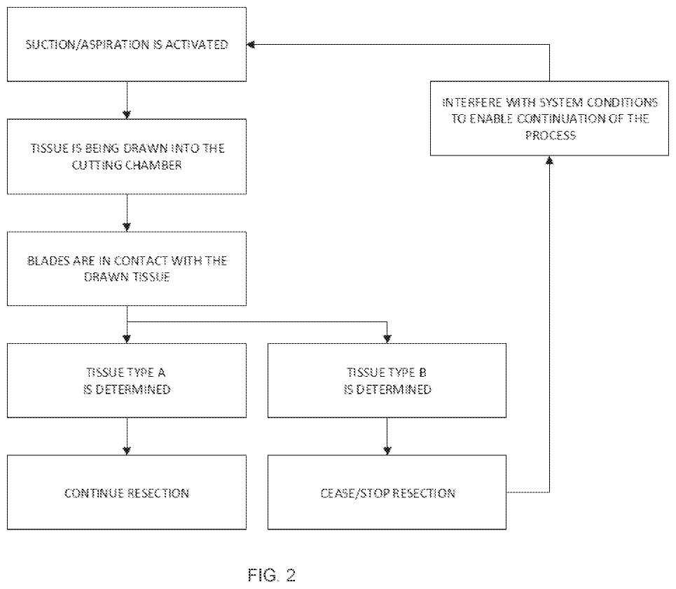

FIGS. 1-2 illustrate the methodology of the system to detect, recognize and selectively resect tissue mass of Type A, while avoiding the resection of tissue mass of Type B, including an optional mechanism of interference to eject tissue that is not intended for resection.

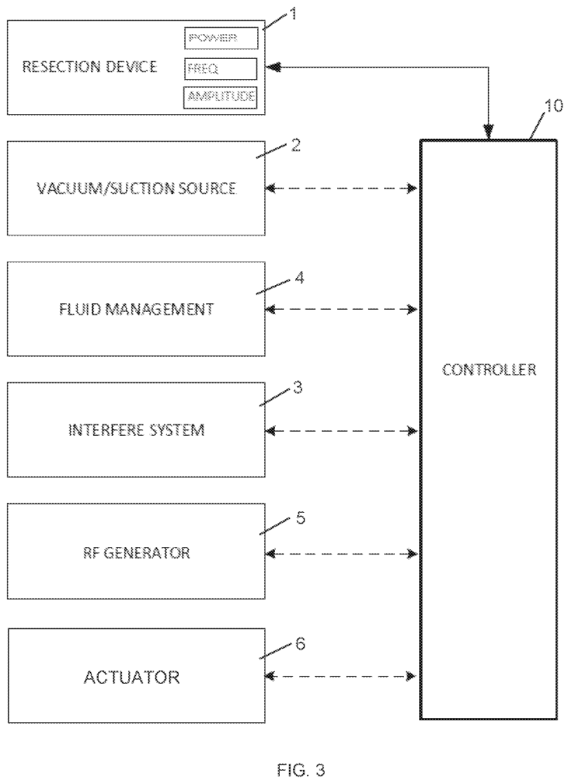

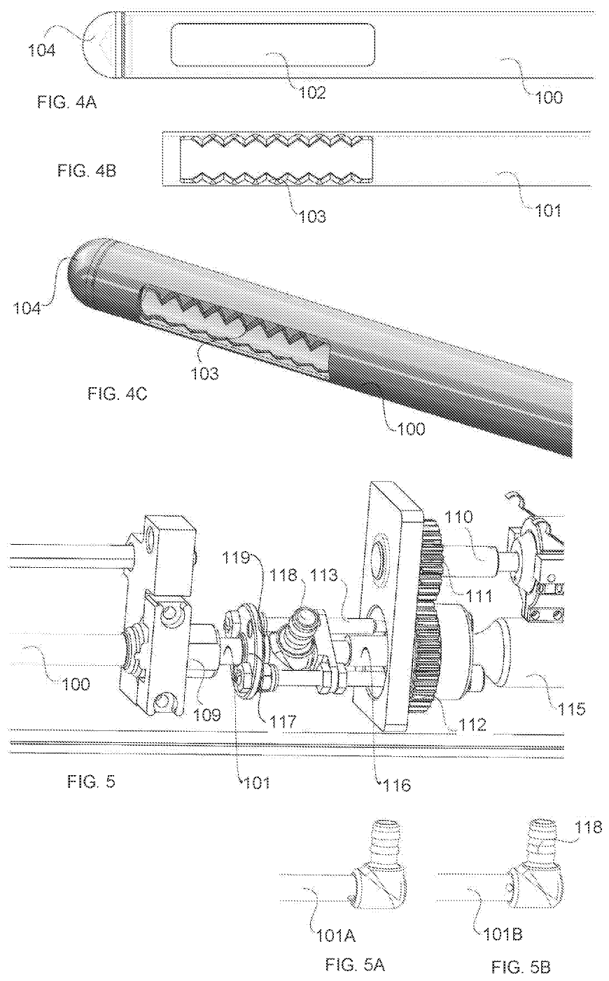

Reference is now made to FIG. 3. In FIG. 3, it is seen that a controller 10 is coupled to a resection device 1 and other additional components including (but not limited to) a vacuum/aspiration source 2, an interference system 3, a fluid management unit 4, an RF generator 5, and an actuator 6, such as a motor, solenoid or other electric, hydraulic or pneumatic actuator for moving the cutting portion of the resection device 1. As will be described with reference to FIG. 5, the actuator may include two actuator portions, one for causing linear oscillation and the other for causing rotational oscillation. The invention is not limited to this, and one actuator may be used for both linear and rotational oscillation.

Specifically, controller 10 is operatively coupled to the cutting portion 103 (shown in FIGS. 4A-4C) of the resection device 1, and may be configured to detect the ability of the cutting portion 103 to resect the tissue.

For example, controller 10 may be connected to actuator 6 which is coupled to cutting portion 103 of the resection device 1. Controller 10 controls operation of actuator 6 to oscillate and rotate the cutting element 103. Controller 10 also may sense the load on actuator 6 as a feedback for detecting a “no-resection” situation between the cutting element 103 and the target tissue, based on physical parameters (e.g., hardness of tissue).

Controller may reverse the direction of rotation of the cutting element (e.g., cutting blades). In one embodiment, this reversed rotation of blades may be configured to cut soft tissue, for example.

In yet another embodiment, the vibration frequency is dynamically changed by the controller to accommodate various tissue cutting configurations, based on the measured force, deflection, deformation, or feedback from the cutting portion or blades.

In yet another embodiment, the controller may change the frequency of the oscillating linear movement to enable or disable tissue cutting of specific types.

In yet another embodiment, the controller may change the amplitude of the oscillating linear movement to enable or disable tissue cutting of specific types.

In yet another embodiment, the controller senses differences in the frequency response when in contact with specific types of tissues.

In yet another embodiment, the controller may also be connected to the vacuum aspiration source to activate or stop the tissue aspiration through the aspiration lumen, and may also be used to read the real-time vacuum levels inside the system for determining a “no-resection” situation between the cutting element and the target tissue.

In yet another embodiment, the controller may be connected to an interference system that may be used to push out tissue (that is not intended for resection) away from the resection window 102 (FIGS. 4A-4C).

In one embodiment, the interference to the suction mechanism can be done by activating a solenoid that injects fluid or pressurized gas in the opposite direction in order to eject the aspirated tissue mass away from the cutting chamber to avoid cutting soft healthy tissue.

In yet another embodiment, if the vibrating blades come into contact with the hard tissue mass, the difference between the measured forces/deflection are expected to be small, the blades cut the hard tissue mass, and the suction/aspiration continues without interruption. When the physician tries to cut soft tissue, the vibrated blades deform the tissue but do not cut (because the soft tissue yields or deflects). The difference between the rotating force exerted by the physician and the responding force of the blades is then above the threshold. The controller senses this difference and ceases the resection process by activating a device that interferes with the suction/aspiration process.

Reference is now made to FIGS. 4A-4C. In one embodiment, a hollow shaft 100 is formed with a window 102 and includes a distal cap 104 at a distal end of the shaft 100. A cutting portion 103 including one or more blades is formed at a distal portion of a tube 101. Tube 101 is disposed inside shaft 100 so that cutting portion 103 is alignable with window 102. The cutting blades may be two parallel rows of cutting teeth, which may be identical or alternatively may be of different shapes and sizes and may be non-parallel.

An oscillating source (e.g., actuator 6) vibrates the cutting portion 103 back and forth in the axial direction. The suction/aspiration unit 2 is connected to the shaft 100 or tube 101 in order to draw a tissue mass inside the cutting chamber 102. When the cutting portion 103 is rotated back and forth, the vibrating blades cut the tissue mass inside the cutting chamber 102. The cut tissue is aspirated by the suction source to an external collector for removal of the undesired tissue mass, fibroid or lesion, and if required, for future histopathology of the removed tissue.

As an option, the window or slit 102 can be partially covered with an outer tube in order to define the length of the dissection.

In yet another embodiment, an injection tube is located inside the window opening 102 (not shown in FIGS. 4A-C) for rejecting tissue away from the opening using a high pressure flow of liquid or gas.

In yet another embodiment, the cutting portion consists of a bent tube, flexible wire (but stiff in the axial direction), or a partially cut lumen tube.

In yet another embodiment, the distal end of the device may include an electrode or trocar for generating RF ablation energy for stopping any bleeding during the procedure.

Reference is now made to FIG. 5 which illustrates an embodiment of resection device. In this version, the actuator includes a linear oscillation actuator 115 and a rotational oscillation actuator 110.

The linear oscillation actuator 115 includes an oscillating piston 116 coupled to tube 101 via a connection member 117, which is secured to a pair of guide rods 113 located on opposite sides of piston 116. As piston 116 slides back and forth (left and right in the sense of FIG. 5), tube 101, connection member 117, and rods 113 also slide back and forth. The linear oscillation may be in a frequency range, without limitation, of 100-100,000 Hz, or in the ultrasonic range.

The rotational oscillation actuator 110 is coupled to the assembly of guide rods 113 via gears 111 and 112. Rotation of actuator 110 causes rotation of rods 113 about the central axis of piston 116, which in turn causes the same rotation of tube 101. The rotational oscillation may be in the range of, without limitation, ±50°.

A swivel suction port 118 may be fluidly connected to the proximal end of tube 101 and may be fluidly sealed at the connection to the tube by a seal (O-ring) 109. The suction port 118 may be used to aspirate the resected tissue. As seen in FIGS. 5A and 5B, tube 101 rotates but swivel suction port 118 does not rotate.

An irrigation tube port 119 may be provided at connection member 117 for injecting irrigation fluid to the resection window 102 (FIGS. 4A-4C). Irrigation port 119 can be used to inject high pressure liquid in order to eject a tissue, not intended for resection, out of the resection window 102 (if needed).

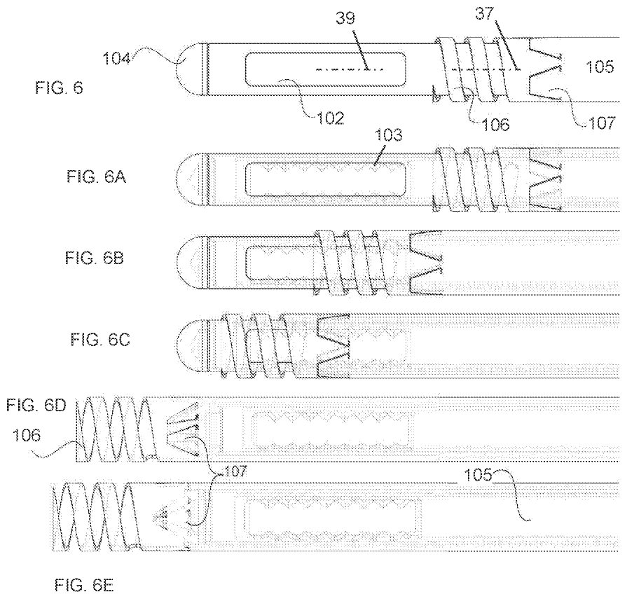

Reference is now made to FIGS. 6-6E which illustrate designs of system and method for preventing unintended tissue perforation. The resection device may include a corkscrew element 106 for a controlled and safe penetration method.

In one embodiment, the corkscrew element cuts or otherwise creates a lumen (which may be straight or curved) in order to create a pathway inside the tissue. This pathway may be used for removal of tissue or debris or for advancing and introducing another medical device.

In yet another embodiment, the corkscrew element and shaft may be used for generating RF ablation energy.

Referring to FIGS. 6-6E, it is seen that the resection device of the invention may include an additional helical (corkscrew) cutting element 106. Accordingly, the resection device of the invention may include a helical cutting element 106 that is disposed around an oscillatory cutting element 103. The helical cutting element 106 may rotate about a rotation axis 37, which is either collinear with or parallel to the longitudinal axis 39 along which the oscillatory cutting element 103 oscillates. The helical cutting element 106 may be arranged to move linearly with respect to the oscillatory cutting element 103 from a position proximal to the oscillatory cutting element 103 (FIGS. 6-6A), to a position that overlies the oscillatory cutting element 103 (FIGS. 6B-6C), and to a position distal to the oscillatory cutting element 103 (FIGS. 6D-6E).

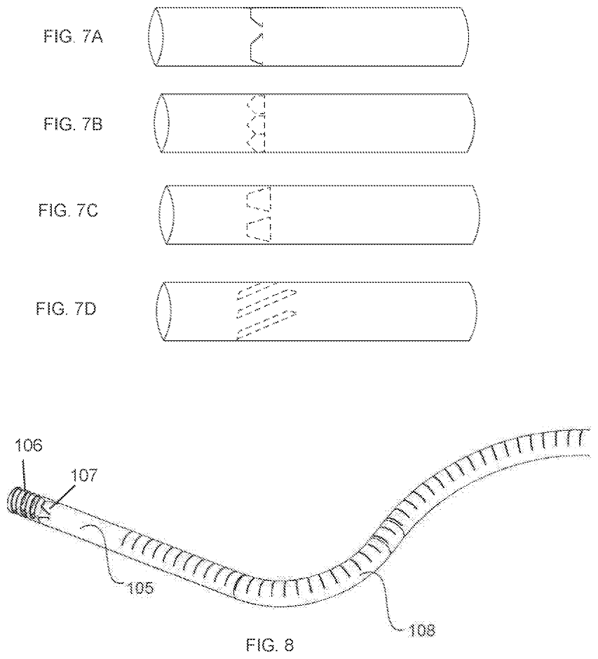

The helical cutting element 106 may extend from a shaft 105 (e.g., a hollow tube). Shaft 105 may have a distal portion which is bendable. For example, shaft 105 may be formed with different cutouts 107 that define areas about which shaft 105 can bend. For example, in FIGS. 6-6E, the cutouts are trapezoidal in shape; in FIG. 7A they are shaped as acute trapezoids (could also be obtuse); in FIG. 7B they are shaped as half-hexagons; in FIG. 7C they are shaped as isosceles trapezoids; in FIG. 7D they are shaped as slanted rectangles. Other shapes are in the scope of the invention.

In FIG. 8, the shaft 105 includes partial circumferential cuts 108 along its axial length (proximal to the cutouts 107), which provide the shaft 105 with further bending capabilities.

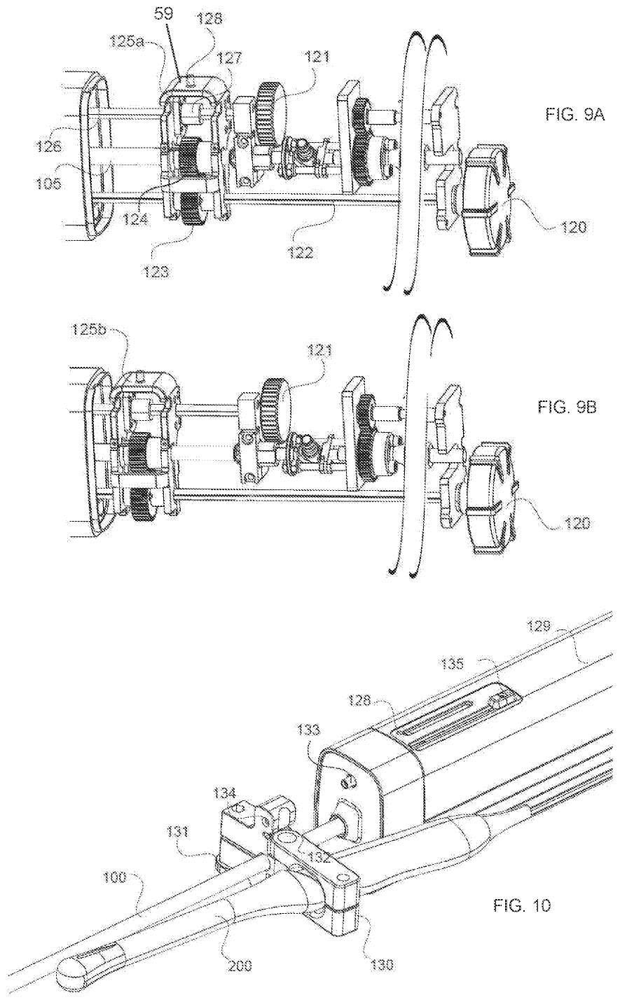

Reference is now made to FIGS. 9A-9B, which illustrate a non-limiting example of actuation of the helical cutting element 106. In the illustrated embodiment, the actuation system includes two actuator portions, one for causing linear advancement or retraction of the element 106 and the other for causing rotation of the element 106. The invention is not limited to this, and one actuator may be used for both linear and rotational motions.

A rotational actuator 120 may be a manual knob or a motor that rotates a connecting shaft 122 coupled to shaft 105 through meshing gears 123 and 124. A linear actuator 121 may be a manual knob or a motor that rotates a bushing 127 along a threaded shaft 126 so that bushing 127, together with a gear cradle 59, move distally or proximally along shaft 126, thereby advancing or retracting shaft 105 and helical cutting element 106. In FIG. 9A, the cradle 59 is at position 125A, in which helical cutting element 106 is proximal to the oscillatory cutting element 103 (FIGS. 6 and 6A). In FIG. 9B, the cradle 59 is at position 125B, in which helical cutting element 106 is distal to the oscillatory cutting element 103 (FIGS. 6D and 6E).

Reference is now made to FIGS. 10-11 which illustrate design of system and method for aligning a resection or RF ablation device 100 to an imaging sensor 200, such as but not limited to, a transvaginal ultrasound probe. The system may be integrated with 2D-3D imaging and planning software to develop and implement a cutting procedure for the particular needs of the patient. Real time feedback can be added to improve accuracy.

Imaging sensor 200 may be coupled to device 100 with an alignment fixture 130. The device 100 can move freely back and forth and also freely rotate inside the fixture 130, until locked at a desired spatial (linear and rotational) orientation with a locking element 131, such as but not limited to, a thumbscrew, locking pin, ratchet and many others. A fastener 132 may be used to clamp imaging sensor 200 at any desired angle with respect to device 100. The locking element 131 can be normally closed (locked) or normally opened (unlocked). In order to align the resection window 102 with the line of sight (bore sight) or imaging plane of the imaging sensor 200, there is a need to fix the orientation of the device 100 compared to the ultrasound probe 200 to avoid situations that the resection window points towards area outside the plane of imaging. This may be achieved by an alignment rod 133 arranged for sliding in a handle 129 by means of a knob 135. Fixture 130 may include an alignment hole 134. By sliding alignment rod 133 from the position in FIG. 10 to that of FIG. 11, the imaging plane of the imaging sensor 200 is considered aligned with the window of device 100 if the alignment rod 133 enters alignment hole 134. After locking the locking element 131, the resection window 102 is aligned towards the proper imaging plane.

Reference is now made to FIG. 12 which illustrates another aligning fixture 242, especially useful if shaft 105 is flexible. Fixture 242 includes a manipulator 136 coupled to helical cutting element 106 by a shaft that passes through, and is lockable relative to, the fixture 242. The tilt angles of the helical cutting element 106 are limited by the manipulator 136 to allow angles only in the plane of the (e.g., ultrasonic) imaging plane (XY plane), thus allowing to visualize and navigate the device shaft 105 in a safe manner, by preventing the helical cutting element 106 from navigating outside the imaging plane, for safety purposes. The manipulator 136 can manipulate the cutting element 106 in multiple degrees of freedom in rotation.

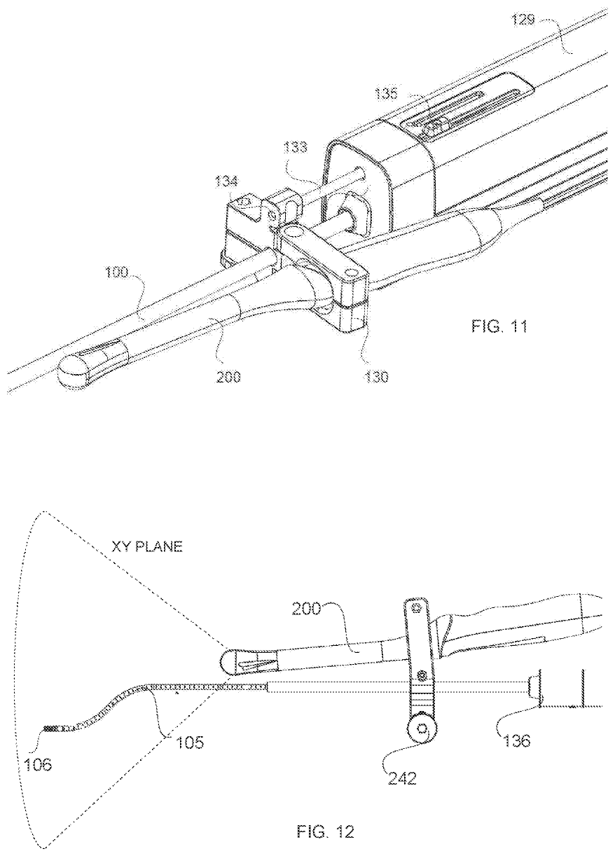

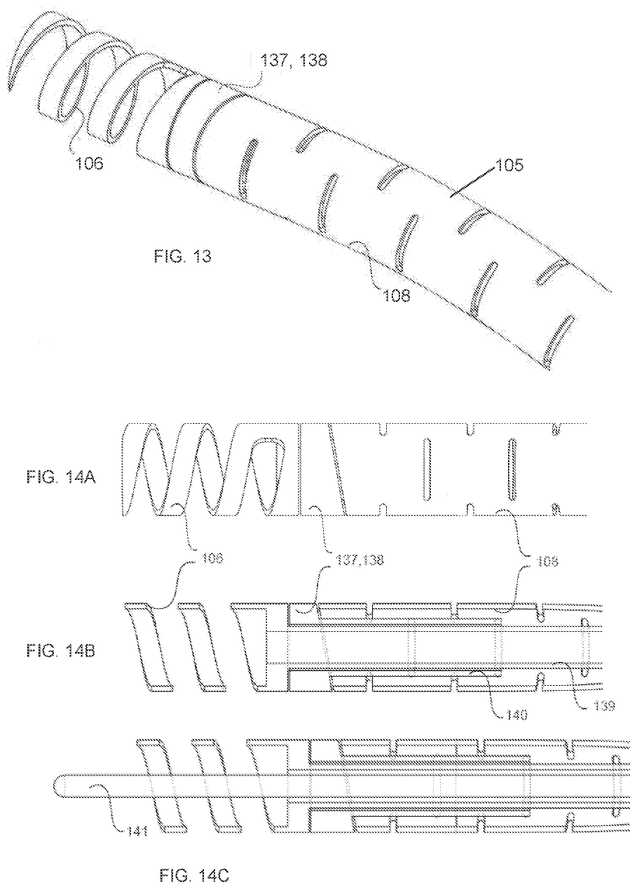

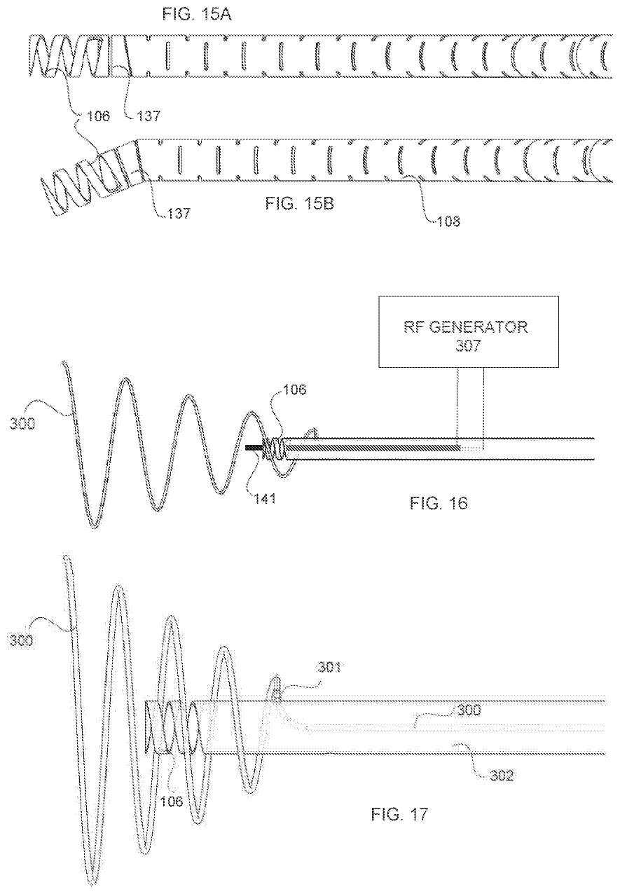

Reference is now made to FIGS. 13-15B, which illustrate one type of coupling between the actuator (such as the actuator in FIGS. 9A and 9B or actuator 136 of FIG. 12) and the shaft 105 of the cutting element 106. As seen in FIG. 13, the flexible portion 108 of the shaft 105 may be coupled to cutting element 106 with a slanted ring 137, that is, the proximal face of the ring is slanted with respect to the distal face of the ring. Slanted ring 137 interfaces with helical cutting element 106 via a coupling 138. Slanted ring may be turned by a distal portion 139 of the actuator which engages a cylindrical shaft 140 that extends proximally from ring 137. FIGS. 15A and 15B show two different rotational orientation of cutting element 106 by appropriate turning of slanted ring 137.

An RF electrode 141 may be used to perform tissue ablation. Both the electrode 141 and the corkscrew element 106 may be used to measure tissue's impedance and/or and tissue temperatures before and during RF ablation process.

In one embodiment, the corkscrew portion 106 is rotated separately without rotating the proximal portion of shaft lumen 108.

In yet another embodiment, the corkscrew actuator 137 causes the corkscrew portion to tilt, independently of the proximal portion of the shaft lumen 108.

In yet another embodiment, the corkscrew element 106 may be made with sharp edges in order to cut or pave a pathway when it is advanced inside the tissue, thus allowing removal of tissue or paving a path inside the tissue to enable another device to advance inside the generated path.

In yet another embodiment, the corkscrew element 106, the shaft 108 or additional electrode 141 may be used for generating RF ablation energy for treating purposes.

Reference is now made to FIG. 16 which illustrates an RF ablation device that includes an expandable electrode that expands as it is advanced deeper into the tissue. In this embodiment, the helical cutting element 106 may further serve as an electrode. An RF generator 307 is electrically coupled to the electrodes 141 and 300, and may also be coupled to helical cutting element 106. In one embodiment, the coil shape of the electrode 300 or the helical cutting element 106 may be used for measuring the ablation temperature during the process or the tissue's impedance for mapping the ablation zone.

Reference is now made to FIG. 17. The proximal portion of expandable electrode 300 is contained inside a lumen shaft or other shaped container 302. Electrode 300 exits lumen shaft 302 through an exit port 301 and then its shape gradually expands radially outwards. Electrode 300 may be gradually advanced into the tissue, forming a larger geometrical volume shape (compared to its initial volume within the container 302) as it is advanced furthermore into the tissue. The final geometrical volume shape of the electrode 300 may be set in advanced by introducing thermal process to the electrode during the manufacturing process to shape the electrode to a pre-defined geometry. Shape memory materials may be used for manufacturing the electrode to give the electrode its final shape.

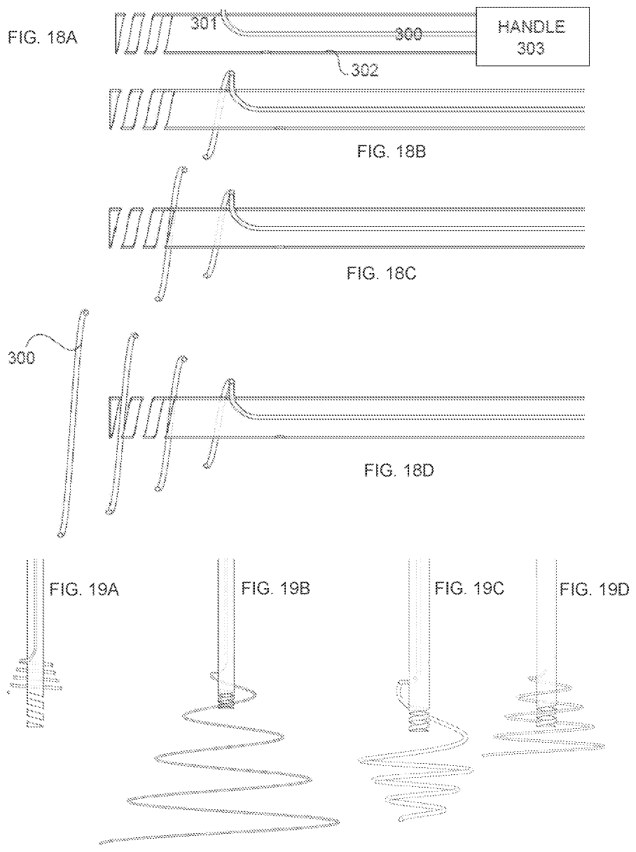

FIGS. 18A-18D illustrate the advancement sequence of the electrode 300. FIG. 18A is the initial state when electrode is fully constrained inside the container 302. The final geometrical shape of the electrode is shown in FIG. 18D. Intermediate states are shown in FIGS. 18B and 18C. A handle 303 may be designed to advance the electrode 300 based on a worm gear or other appropriate mechanism.

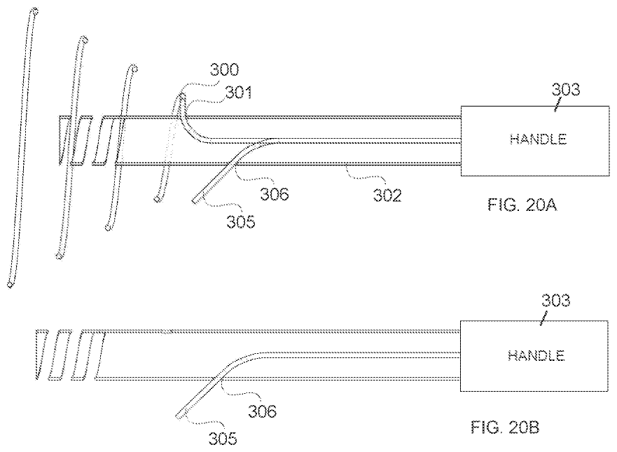

Reference is now made to FIGS. 19A-19D, which illustrate examples of different sizes, shapes, thicknesses and cross sections of the electrode. In one embodiment, the shape of the electrode may be of symmetric or asymmetric shape, with variable cross sections, coil pitch, and coil amplitudes/diameters. The cross section may circular or flat or other shape.

Reference is now made to FIGS. 20A-20B, which illustrate examples of plural electrodes exiting from port holes such as 301 and 306 without limiting the number of electrodes or port locations. In yet another embodiment, the plural electrodes may be of different shapes and sizes, so the superposition of all electrodes may generate and form a desired 3D volume of the ablation area in the tissue.

What is claimed is:

1. A surgical device comprising:
a housing formed with a window;
a cutting element disposed in said housing and coupled to a vibration source, said vibration source operative to cause said cutting element to oscillate;
an imaging sensor; and
an actuator coupled to said housing or to said cutting element and in operative communication with said imaging sensor.

2. The surgical device according to claim 1, wherein said actuator is operative to align said window with an imaging direction of said imaging sensor.

3. The surgical device according to claim 1, comprising a directional suction source configured to draw tissue into said housing, wherein said actuator is operative to align said directional suction source with the plane of an imaging direction of said imaging sensor.

4. The surgical device according to claim 1, wherein said cutting element is movable in a linear motion.

5. The surgical device according to claim 1, wherein said cutting element is movable in a non-linear motion.

6. The surgical device according to claim 1, wherein said housing is coupled to a rotatable helical element, wherein rotation of said helical element causes linear movement of said housing.

7. The surgical device according to claim 1, wherein said helical element is coupled to a bendable member, such that said helical element is movable along a non-linear path in response to bending of said bendable member.

8. The surgical device according to claim 1, further comprising a tissue hardness detector coupled to said vibration source.

9. The system according to claim 8, wherein if hardness is not above a threshold, said vibration source decreases a vibration amplitude so as not to permit tissue cutting.

10. The system according to claim 3, further comprising a tissue hardness detector, wherein if hardness is not above a threshold, said tissue hardness detector actuates an interference device that interferes with said suction source and does not permit said suction source to draw tissue into said housing.

11. The system according to claim 10, wherein said interference device comprises a solenoid that injects liquid or pressurized gas that opposes suction of said suction source so as to eject the tissue away from said housing.

12. The surgical device according to claim 1, wherein said helical element is slidable over a shaft coupled to said cutting portion.

13. The surgical device according to claim 1, wherein helices of said helical element are expandable radially outwards.

14. The surgical device according to claim 1, wherein said helical element is coupled to said imaging sensor or to another imaging sensor.

15. The surgical device according to claim 1, comprising an actuator sensor configured to measure a change or deflections in a vacuum load level of said cutting element.

16. The surgical device according to claim 1, comprising an actuator sensor configured to measure a change in a load of said actuator.

17. The surgical device according to claim 1, comprising an actuator sensor configured to measure a change in mass flow at or near said cutting element.

18. The surgical device according to claim 1, comprising an actuator sensor configured to measure a difference between forces, deflections or power of said cutting element compared to forces, deflections or power of said actuator.

19. A surgical device comprising:
a helical cutting element disposed around an oscillatory cutting element.

20. The surgical device according to claim 19, wherein said helical cutting element rotates about a rotation axis which is either collinear with or parallel to a longitudinal axis along which said oscillatory cutting element oscillates.

21. The surgical device according to claim 19, wherein said helical cutting element is movable linearly with respect to said oscillatory cutting element from a position proximal to said oscillatory cutting element to a position that overlies said oscillatory cutting element, and to a position distal to said oscillatory cutting element.

22. A system for ablation comprising:
a helical member coupled to a housing member and configured to move and position said housing member in a tissue, a portion of said helical member having a side aperture; and
a flexible member deployable through said side aperture, said flexible member being capable of assuming a helical shape and transmitting RF energy to the tissue.

23. The system according to claim 22, wherein said flexible member has a variable cross section.

24. The surgical device according to claim 22, wherein a portion of said helical member is coupled to a bendable member, wherein said bendable member is operative to cut a route in the tissue in accordance with bending of said bendable member.

25. The system according to claim 22, comprising an actuator coupled to said helical member and in operative communication with an imaging sensor, wherein said actuator is operative to align said helical member with an imaging direction of said imaging sensor.
